(19) United States
(12) Patent Application Publication
(10) Pub. No.: US 2025/0275573 A1
(43) Pub. Date: Sep. 4, 2025

(54) ACOUSTOFLUIDIC DEVICE FOR GENERATING AEROSOLS

(71) Applicant: Leibniz-Institut für Festkörper- und, Werkstoffforschung Dresden e. V., Dresden (DE)

(72) Inventors: Iman FROZANPOOR, Dresden (DE); Mehrzad ROUDINI, Berlin (DE); Yara ALSAADAWI, Dresden (DE); Andreas WINKLER, Dresden (DE)

(73) Assignee: Leibniz-Institut für Festkörper- und, Werkstoffforschung Dresden e. V., Dresden (DE)

(21) Appl. No.: 19/066,845

(22) Filed: Feb. 28, 2025

(30) Foreign Application Priority Data
Mar. 1, 2024 (DE) 10 2024 105 991.5

(51) Int. Cl. A24F 40/10; A24F 40/42; A24F 40/46; A24F 40/48; A24F 40/51

(52) U.S. Cl. CPC A24F 40/10; A24F 40/42; A24F 40/46; A24F 40/48; A24F 40/51

(57) ABSTRACT

An acoustofluidic device usable, e.g., in devices for printing. The acoustofluidic device has small dimensions and can be integrated into devices for the deposition of aerosols and, with a simple design, realizes a directed aerosol flow with adjustable aerosol properties. The acoustofluidic device at least includes at least one aerosol discharge component and at least one holder component and at least one feed and/or removal line each for gases, liquids, and heat, at least one electrical line and/or electrical signal line and at least one electrical high-frequency signal line and at least one component for heat transport and at least one temperature sensor and at least one microacoustic component and at least one component for the transmission of a high-frequency signal and at least one component for liquid transport and at least one transport gas component and at least one component for positioning and/or mechanical fixing.

### CROSS-REFERENCE TO RELATED APPLICATION

This application claims priority under 35 U.S.C. § 119 (a) of German Application No. 10 2024 105 991.5 filed Mar. 1, 2024, the disclosure of which is expressly incorporated by reference herein in its entirety.

The invention pertains to the fields of microsystems technology, microfluidics, and microacoustics and relates to an acoustofluidic device for generating aerosols which can be used, for example, in devices or systems for the deposition of aerosols, or in devices and print heads for the printing of or by means of aerosols (aerosol jet printing), or in chemical or physical systems for material deposition, materials synthesis or material etching, such as atomic layer deposition systems (ALD), atomic layer etching systems (ALE), physical vapor deposition systems (PVD), chemical synthesis systems or chemical vapor deposition systems (CVD), pyrolysis systems, and can be used in aerosol sources for humidifiers, spray drying and spray cooling, and can be used and applied, for example, in the fields of life sciences, bioanalysis, biochemistry, chemistry, medical engineering, materials science, thin-layer technology, coating technology, or in the printing industry.

Microsystems technology is a branch of microtechnology and concerns the science, research, development, and production of microsystems. For example, micromechanical or micro-optical components with microelectronic circuits are combined and integrated in a complex system. Sensors, actuators, and data processing work together in microsystems. Processes and methods from different microtechnologies are therefore combined in order to produce complex microsystems. These include micromechanics, microfluidics, bioelectronics, micro-optics, and microelectronics (data processing, electronic interfaces) itself. The production methods used are very diverse: In addition to the typical thin-layer technologies, molding technologies such as lithography, electroplating and molding (LIGA), etching technologies, etc. are also used. Microsystems technology uses virtually all types of materials such as metals, semiconductors, ceramics, sol-gel materials, plastics, and many more.

Microfluidics deals with the behavior of liquids and gases in extremely small spaces (Wikipedia, German-language keyword “Mikrofluidik”).

Acoustics is the science of sound (Wikipedia, German-language keyword “Akustik”).

Surface acoustic waves (SAW) are solid-state waves which propagate in a planar manner on a surface, that is, in only two dimensions (Wikipedia, German-language keyword “akustische Oberflächenwelle”). In addition to SAWs, there are other solid-state acoustic waves which can be excited on chip substrates, for example, plate acoustic waves, edge acoustic waves, boundary layer acoustic waves, and volume acoustic waves.

Aerosols are known as heterogeneous mixtures (dispersions) of fine solid and/or liquid particles in a gas (Wikipedia, German-language keyword “Aerosol”). Aerosols that contain only liquid particles in a gas are also referred to as mist or vapor.

Aerosol jet printing (AJP) is a contactless direct writing technology which applies liquids with high precision to different substrates and enables the printing of fine electronic, structural, and biological patterns. The German-language “Aerosolstrahl” [aerosol stream] can also be referred to as “Aerosoljet” [aerosol jet].

Chemical or physical synthesis systems are known as systems which apply gas and/or aerosol flows to a substrate, for example a wafer, in a defined atmosphere, for example a vacuum or an inert gas atmosphere, in order to obtain layer growth or structure growth or the removal of a layer (etching). The known methods include physical gas phase deposition (PVD, for example sputtering or vaporization); chemical gas phase deposition (CVD), also in combination with plasma, ion, or microwave sources; atomic layer deposition (ALD); reactive ion etching (RIE) or atomic layer etching (ALE).

Due to the increasing complexity of technical systems, an increasing number of components are being integrated into electronic circuits and utilized for numerous novel applications, in particular in microsystems technology. In particular, components from microfluidics and/or components which generate and/or utilize surface acoustic waves are integrated into systems of this type, which results in a smaller device footprint, higher efficiency, and significantly broader and novel areas of application. Similarly, components which generate or utilize surface acoustic waves are increasingly being used in technology, which components can also be used in combination with microfluidic components, and are in this case referred to as acoustofluidic components.

Known areas of application for microsystems technology with aerosols are, in particular, aerosol jet printing and chemical deposition (ALD, CVD), in particular involving the use of a transport gas with a chemical precursor.

In the aerosol jet printing (AJP) method, a liquid phase, such as a metal particle dispersion for example, is atomized into droplets, referred to as aerosol ink, which is then transferred onto a surface through a nozzle with the aid of a focusing gas sheath. Aerosol jet printing is a direct writing method for high-resolution, maskless material deposition of structures in the size range of approximately 10 μm to 5 mm. In this method, the aerosol generated by an aerosol generator is spatially focused by means of a gas flow, and is applied at a distance in the cm range via a nozzle to a surface that is to be coated. The AJP method is already being used in the field in the area of printed electronics, for example in the production of integrated antennas (Godlinski, D. et al: IET Microw. Antennas Propag. Article vol. 11, no. 14, pp 2010-2015, November 2017), for chip packaging and for high-frequency contacts (https://optomec.com/applications/aerosol.jet), in the production of front contacts for solar cells (Binder, S.: Aerosol Jet Printing, Technische Universität Ilmenau 2017) and in the printing of conductor paths on 3D-printed components and freeform components (http://investors.stratasys.com/news-events/press-releases/detail/177/3d-printing-is-merged-with-printed Elektronik). Other research approaches deal with electronic mounting and connection technology (SMD, MID) (Krzeminski, J. et al: IEEE Trans. Nanotechnol. Article, Proceedings Paper vol. 17, no. 5, pp 979-984, September 2018), with conductor path integration into aeronautics and space structures, with the printing of photodiodes and the printing of piezo sensors. AJP is also increasingly being used for the printing of biological materials.

According to the prior art, the aerosol generation for aerosol jet printing is currently realized by means of pneumatic or ultrasound methods which are embodied as separate aerosol chambers. The generated aerosol ink is then conducted by means of a carrier gas flow from the aerosol chamber into the actual print head, where it is collimated by means of a coaxial focusing gas flow, directed onto the substrate that is to be coated, and deposited there. Due to the complex system architecture of current AJP designs, it is not possible to integrate all aerosol carrying, gas carrying, and liquid carrying components into one compact print head, whereby a high cost for the setup and cleaning of the aerosol chamber, of the supply lines, and of the print head arises during operation. In the case of cost-intensive printing liquids or those which are only available in small amounts, the material loss in the supply lines or as a result of large start/dead volumes (several ml) also constitutes a considerable problem.

In the pneumatic atomization for aerosol jet printing, a carrier gas is accelerated via the liquid-gas boundary, whereby a region with reduced static pressure arises which draws the liquid into the carrier gas flow. The instability of the thin liquid layers, which results from the interaction with the surrounding gaseous medium, leads to rapidly growing (capillary) waves at the liquid-gas boundary. The degradation of the liquid occurs when the wave amplitude reaches a critical value. Fragments of the layers are stripped off and, under the effect of the surface tension, quickly contract to form unstable bands, and when said bands collapse, drops form.

Pneumatic atomizers have the disadvantage that they must be operated at high pressure via an external air compressor, and that they exert high shearing forces on the liquids. This can damage sensitive liquids and additionally results in a broad droplet size distribution, which reduces the resolution and quality of the deposited structures. The very broad droplet size distribution also results in a low aerosol yield, and therefore in a lower deposition rate, and requires the use of a virtual impactor/aerosol separator for the separation of larger aerosol droplets and for pressure reduction.

Furthermore, ultrasound volume transducers are used for the generation of aerosols for aerosol jet printing. With these devices, a liquid is acoustically excited by an ultrasound transducer. In the reservoir, (capillary) waves are generated at the liquid-gas boundary and cause the liquid to degrade into an aerosol made of droplets of a differing size, which aerosol is then removed using a transport gas flow. However, this solution has several considerable disadvantages, such as a large, unwieldy device, a high dead volume, and therefore sample losses, as well as very high cleaning cost. Ultrasonic atomizers are only suitable for liquids with a relatively low viscosity of 1-20 mPa·s. This limits the material selection for the liquids, since in particular many biological liquids have a higher viscosity and cannot be atomized at all in this manner.

For a homogeneous print image and continuous printing, the liquid level in the aerosol chamber should also be kept constant during ultrasound-based aerosol generation, which requires a very complicated refill construction, however.

In both technologies (pneumatic and ultrasound), the continuous recirculation of the aerosol inks in the aerosol chamber leads to the vaporization of the readily volatile solvents in the liquids and alters the composition thereof, which likewise has a negative effect on the print quality and reduces the aerosol yield.

In addition to the aforementioned disadvantages of individual solutions, the known solutions from the prior art have the disadvantage that the dimensions of the devices and components for generating aerosols are too large, and that said devices and components therefore cannot be integrated into other devices without limitations, nor is it possible to reduce and miniaturize the dimensions of said devices and components themselves. Likewise, it is not yet possible to adjust with adequate precision different properties of the aerosol, such as the geometry or the velocity of the aerosol stream and the temperature of the liquid, within broad limits.

Solutions for SAW-based aerosol generation are already known (Roudini, M. et al: J of Aerosol Science, Jul. 18, 2023). The SAW aerosol generating chips have focused and straight interdigital transducers (IDTs) and integrated microfluidic liquid feed channels which are produced on a wafer scale by multi-stage dry-film lamination and lithographic structuring technologies.

The object of the present invention is to provide an acoustofluidic device for generating aerosols, that is an aerosol generator assembly, which has small dimensions and can be integrated into devices or systems for the deposition of aerosols and, with a simple design, realizes a directed aerosol flow with adjustable aerosol properties.

The object is attained by the invention recited in the claims. Advantageous embodiments are the subject matter of the dependent claims, wherein the invention also includes combinations of the individual dependent claims within the meaning of an AND-combination, provided that they are not mutually exclusive.

The acoustofluidic device for generating aerosols according to the invention at least contains:
at least one aerosol discharge component with at least one opening from which an directed aerosol stream or jet is discharged; and
at least one holder component for at least one microacoustic component; and
at least one feed and/or removal line for gases, liquids, and heat, at least one electrical line and/or electrical signal line, and at least one electrical high-frequency signal line; and
at least one component for heat transport, which realizes the transport of heat from and/or to at least one holder component and which is connected to at least one feed and/or removal line for heat; and
at least one temperature sensor which is connected in a thermally conductive manner to the holder component and/or to the heat transport component and in an electrically conductive manner to at least one electrical high-frequency signal line; and
at least one microacoustic component having a lateral dimension of maximally 4×4 cm² with at least one acoustic transducer and/or electroacoustic and/or piezoelectric transducer for the excitation of acoustic waves in the frequency range of 1 to 500 MHz; and
at least one component for the transmission of a high-frequency signal which is connected in an electrically conductive and high-frequency suitable manner to at least one electrical high-frequency signal line and to at least one microacoustic component, and which realizes the transmission of a high-frequency signal to and/or from the at least one microacoustic component via the at least one electrical high-frequency signal line; and
at least one component for liquid transport which is connected in a liquid conducting manner to at least one feed and/or removal line for liquids and to at least the one microacoustic component, and with which at least the feed of liquids to the surface of the at least one microacoustic component is realized at a volume flow rate between 0.1 μl/min and 5 ml/min; and
at least one transport gas component which is connected to at least one feed and/or removal line for gases, wherein with the transport gas component, at least the feed of at least one gas into the immediate proximity of the region of the acoustofluidic interaction between the acoustic wave and the liquid for the aerosol generation is realized on the surface of the microacoustic component to which the liquid is applied; and
at least one component for positioning and/or mechanically fixing the at least one component for the transmission of a high-frequency signal and the at least one component for liquid transport in relation to the at least one microacoustic component or to the at least one holder component, wherein the at least one component for positioning and/or mechanical fixing is a standalone component and/or a components of the holder component and/or of the component for liquid transport and/or of the aerosol discharge component and/or of another component.

Advantageously, the at least one microacoustic component realizes the excitation of at least one acoustic wave, advantageously of a Rayleigh surface acoustic wave and/or of a Sezawa surface acoustic wave; and/or of a plate acoustic wave, advantageously of a symmetrical and/or asymmetrical Lamb wave; and/or of a volume acoustic wave, advantageously of a longitudinal and/or shear wave; and/or of a standing surface acoustic wave and/or of a solid-state and/or liquid-state acoustic wave.

Also advantageously, the at least one component for liquid transport is connected to at least one liquid pump and/or at least one liquid reservoir, or contains at least one liquid pump and/or at least one liquid reservoir and/or at least one micropump and/or at least one electroosmotic pump and/or at least one liquid valve and/or at least one sensoric and/or analytical component.

Further advantageously, at least one component for positioning and/or mechanical fixing realizes a reversible vertical tilting in the sense of a partial rotation or a reversible vertical raising in the sense of a displacement or a reversible sliding at least of the at least one component for the transmission of a high-frequency signal and of the at least one component for liquid transport in relation to the at least one microacoustic component or to the at least one holder component for the purpose of replacing the microacoustic component, wherein it is more advantageous if the reversible installation of the at least one microacoustic component is realized by a lateral sliding, insertion, or displacement of the microacoustic component into or onto the holder component, or by vertical placement or insertion of the microacoustic component into or onto the holder component.

Likewise advantageously, the at least one heat transport component contains or is connected to at least one heat pipe and/or a cooling body with or without components for improving the gas convection and/or with liquid cooling connected thereto and/or at least one Peltier element having a passive finned cooler with or without components for improving the gas convection and/or at least one Peltier element having a cooling body with liquid cooling connected thereto.

It is also advantageous if the at least one microacoustic component is a microacoustic chip or a microacoustic cartridge, wherein the microacoustic cartridge is at least composed of at least one microacoustic chip on at least one plate or film of a material with high thermal conductivity, and wherein additional layers of highly thermally conductive material are present at least between the chip and plate or film and/or on the side of the plate or film facing away from the chip, wherein it is more advantageous if a highly thermally conductive connection is realized between the microacoustic chip and the plate or film using an adhesive and/or an adhesion-promoting liquid, and/or single- or double-sided adhesive tape, and/or metal- and/or ceramic-filled polymers, epoxies and/or pastes, and/or an adhesive tape or adhesive strip, and/or by means of a non-permanent adhesive.

It is further advantageous if at least one microacoustic component and/or at least one component for liquid transport comprises at least one microfluidic and/or liquid conducting structure element and the at least one component for liquid transport is connected in a liquid conducting manner to the microacoustic component via at least one microfluidic and/or liquid conducting structure element, wherein it is more advantageous if at least one microchannel and/or one connecting element and/or one sealing element and/or one reservoir and/or one micromembrane and/or one needle-shaped structure element and/or one sponge- or tissue-shaped element is present as a microfluidic and/or liquid conducting structure element.

It is likewise advantageous if at least one transport gas component realizes a coaxial inflow of a gas into the region of the acoustofluidic interaction between the acoustic wave and the liquid for the purpose of sheathing the generated aerosol stream or jet, wherein the site of the discharge of the gas from the transport gas component is arranged maximally 30 mm, advantageously <15 mm, more advantageously <5 mm, away from the liquid carrying surface of the at least one microacoustic component in the direction of the volume flow vector of the aerosol stream or jet, wherein it is more advantageous if the rotationally symmetrical volume flow vector of the coaxially inflowing gas deviates by no more than 45°, advantageously by no more than 30°, from the volume flow vector of the aerosol stream or jet, or from the surface normal of the liquid carrying surface of the at least one microacoustic component.

And it is also advantageous if, downstream from the at least one transport gas component or after the at least one opening in the aerosol discharge component in the direction of the volume flow vector of the aerosol stream or jet, at least one component is present for the aerodynamic focusing of the aerosol stream or jet with a gas sheath and/or for increasing the velocity of the aerosol stream or jet, which component comprises at least one additional feed and/or removal line for gases.

It is furthermore advantageous if an additional, tubular enclosure surrounds the acoustofluidic device, which enclosure is composed of metal, polymer, ceramic, or glass and which comprises on at least one tube end at least one flange of the type ISO-K, ISO-KF, ISO-F, CF, or COF for the feed and removal lines and electrical signal lines and electrical high-frequency signal lines.

It is likewise advantageous if the opening or the openings in the aerosol discharge component have dimensions of no greater than 5 mm, and/or have a circular, coaxial, and/or oval shape, and/or are multi-opening outlets, and/or have Luer threads and/or metric threads and/or inch threads.

It is also advantageous if at least one open-loop and/or closed-loop control component is present for the open-loop and/or closed-loop control of the discharge of the aerosol stream or jet from the aerosol discharge component or from the component for the aerodynamic focusing of the aerosol stream or jet, which control component is arranged on the outer side or inner side of the aerosol discharge component or of the component for the aerodynamic focusing of the aerosol stream or jet or is arranged downstream above the microacoustic component and/or is part of the component for liquid transport, wherein it is more advantageous if the open-loop and/or closed-loop control component is a rotatable or movable bar that is positioned in the aerosol stream or jet and/or is a component for closing the opening or the openings and/or is a component which contains a suction device.

The present invention renders it possible for the first time to specify an acoustofluidic device for generating aerosols which has small dimensions and which can be integrated into devices or systems for the deposition of aerosols and which, with a simple design, realizes a directed aerosol flow with adjustable aerosol properties.

This is achieved with an acoustofluidic device for generating aerosols in which the aerosol generation is achieved by the interaction of the liquid that is to be atomized with a high-frequency acoustic wave which is generated by at least one microacoustic component. The atomization by means of a high-frequency acoustic wave results in aerosols with very small droplets and a very homogeneous droplet size distribution, which can be reproducibly and dynamically adjusted in terms of the droplet size. With the at least one microacoustic component, it is also possible to realize a wide parameter range of flow rates. In addition, the properties of the aerosol stream (velocity, width, and angle) can be adapted, which enables a more efficient focusing of the aerosol stream.

The acoustofluidic device for generating aerosols according to the invention contains at least one aerosol discharge component, at least one holder component, at least feed and/or removal lines for gases, liquids, and heat, respectively, at least one heat transport component, at least one temperature sensor, at least one microacoustic component, at least one component for the transmission of a high-frequency signal, at least one component for liquid transport, at least one transport gas component, and at least one component for positioning and/or mechanical fixing.

In accordance with the present invention, the at least one aerosol discharge component according to the invention has at least one opening from which a directed aerosol stream or jet is discharged.

It is advantageous if the at least one opening in the aerosol discharge component from which a directed aerosol stream or jet is discharged has dimensions no greater than 5 mm and/or has a circular, coaxial, and/or oval shape and/or is multi-opening discharge s and/or has Luer threads and/or metric threads and/or inch threads.

According to the invention, there is at least one feed and/or removal line each for gases, liquids, and heat, and at least one electrical line and/or electrical signal line and at least one electrical high-frequency signal line.

For the integration into higher-level systems, for example chemical or physical synthesis systems, it is advantageous if the acoustofluidic device according to the invention is present such that it is installed in a tubular enclosure which can be composed of metal, polymer, ceramic, or glass and which comprises on at least one tube end at least one flange, for example of the type ISO-K, ISO-KF, ISO-F, CF, or COF, for the feed and removal lines and electrical signal lines and electrical high-frequency signal lines.

Furthermore, at least one holder component for at least one microacoustic component is present according to the invention.

In addition, the acoustofluidic device according to the invention has at least one heat transport component which realizes the transport of heat from and/or to the at least one holder component. The heat transport component is connected to at least one feed and/or removal line for heat.

It is advantageous that the holder component is at least partially composed of a material with high thermal conductivity. One or more additional layers of thermally conductive material can also be located between the heat transport component and the holder component.

For the components of the acoustofluidic device according to the invention, a plurality of materials can be used, for example metallic or non-metallic material, polymers, in particular printable or CNC-millable polymers or polymer-containing composite materials, glass, oxides, ceramics, or composites. The components can respectively be composed of a single material or of multiple different materials and/or components. Likewise, the combining/joining of individual components of the acoustofluidic device according to the invention in a combined component is possible, such as the combination of the aerosol discharge component and the transport gas component, for example.

The at least one heat transport component according to the invention prevents temperature fluctuations of the holder component and temperature deviations from a target value of the holder component, and therefore of the liquids which are to be atomized to form an aerosol. Consistent conditions for the aerosol generation can thus be ensured, and temperature systems can be adapted to the requirements of the liquids and/or of the printing process.

The at least one heat transport component can at least comprise the following: a heat pipe and/or a cooling body with or without components for improving the gas convention and/or the liquid cooling connected thereto and/or at least one Peltier element having a passive finned cooler with or without components for improving the gas convection and/or at least one Peltier element having a cooling body with a connected liquid cooling.

A Peltier element is an electrothermal transducer which produces a temperature difference with the aid of a current flow on the basis of the Peltier effect. Peltier elements can be used for both cooling and heating (Wikipedia, German-language keyword “Peltier-Element”).

If the liquid cooling is part of the heat transport component, it can be flexible or rigid tubes, pipes, channels, or other liquid conducting elements. They can be used to alter and control the temperature of the heat transport component and/or of the Peltier element, and therefore the temperature of the holder component, and in particular to increase or decrease the temperature of the holder component or of the Peltier element to a target value.

According to the invention, at least one temperature sensor is provided for the detection of the temperature of the holder component and/or of the temperature of the heat transport component, which temperature sensor is connected in a thermally conductive manner to the holder component and/or to the heat transport component, and is connected in an electrically conductive manner to at least one electrical signal line.

According to the invention, there is at least one microacoustic component having a lateral dimension of maximally 4×4 cm² with at least one acoustic transducer and/or electroacoustic and/or piezoelectric transducer for the excitation of acoustic waves in the frequency range of 1 to 500 MHz.

The term microacoustics is used here to describe systems which are based on the excitation, propagation, and/or interaction of acoustic waves with a typical wavelength of less than one millimeter. This means that microacoustic systems are based on working frequencies above the audible spectrum.

According to the invention, the at least one microacoustic component comprises a surface region in which an interaction between the acoustic wave and the fluid is realized for the aerosol generation.

Surface acoustic waves, advantageously Rayleigh surface acoustic waves and/or Sezawa surface acoustic waves; and/or plate acoustic waves, advantageously symmetrical and/or asymmetrical Lamb waves; and/or volume acoustic waves, advantageously longitudinal and/or shear waves; and/or standing surface acoustic waves; and/or solid-state and/or liquid-state acoustic waves can be excited in the microacoustic component as acoustic waves. More advantageously, Rayleigh standing surface acoustic waves can be excited.

Advantageously, the at least one microacoustic component can be a microacoustic chip or a microacoustic cartridge, wherein the microacoustic cartridge is at least composed of at least one microacoustic chip on at least one plate or film of a material with high thermal conductivity. The plate or film to which the microacoustic chip is attached can, for example, be made of metal, polymer, glass, ceramic, or a composite material, and/or can also be composed of a multilayer plate. The plate or film can also comprise guide elements for the attachment to the holder component. Likewise, the plate or film can, on the side on which it is contact with the holder component in the acoustofluidic device, comprise a thermally conductive layer.

The microacoustic component and in particular a microacoustic chip have at least one acoustic transducer and/or electroacoustic and/or piezoelectric transducer which is electrically connected to at least one component for the transmission of a high-frequency signal line.

Advantageously, the transducer of the microacoustic component is an interdigital transducer made of a structured metal thin film.

The microacoustic component and in particular a microacoustic chip can advantageously comprise a piezoelectric material and/or at least one layer of a piezoelectric material on a non-piezoelectric material and/or at least one non-piezoelectric material on a piezoelectric material. Advantageously present as materials for the microacoustic component are non-piezoelectric materials such as glass, ceramic, glass/ceramic composites, composites with SiO₂, Al₂O₃, Si₃N₄, TIN, SiN, or borosilicate glass; or such as metals/metal alloys such as Al, Cu, Ti, Ta, TiAl, CuTi; or polymers such as PMMA, PTFE, PEEK, polyimide, PET, COP, PDMS, PC, COC, polycaprolactone, PS; or photoresists such as SUEX, ADEX, TMMF S2045, Ordyl, SU-8; or semiconductor materials such as Si, GaAs, InAs, GaN; and/or piezoelectric materials such as quartz, LiNbO₃, black LiNbO₃, yellow-black LiNbO₃, LiTaO₃, AlN, Sc-AlN, ZnO, TaGa₃, CTGS, SiO₂, langasite, gallium orthophosphate, PZT, PMN-PT, or PVDF; or combinations of said materials.

In addition, between the microacoustic chip and the plate or the film of a microacoustic cartridge and between the plate or film of a microacoustic cartridge and the holder component, advantageously one or more additional layers of a highly thermally conductive material can be arranged, which layers can be composed of adhesive and/or adhesion-promoting liquid, and/or can be single- or double-sided adhesive tape, metal- and/or ceramic-filled polymers, epoxies and/or pastes, and/or an adhesive tape or adhesive strip, and/or a non-permanent adhesive. Example materials thereof are silicones with graphite or ceramic particles and with woven carbon fibers or other woven fabrics, as well as silicone-free soft polymers having the aforementioned fillers or woven fabrics or gap-filler materials.

Furthermore, according to the invention at least one component for liquid transport is provided which is connected in a liquid conducting manner to at least one feed and/or removal line for liquids and to the at least one microacoustic component, and with which at least the feed of liquid to the surface of the at least one microacoustic component on which the interaction of the liquid with the acoustic wave occurs is realized at a volume flow rate between 0.1 μl/min and 5 ml/min.

Advantageously, the at least one component for liquid transport is connected to at least one liquid pump and/or at least one liquid reservoir, or contains at least one liquid pump and/or at least one liquid reservoir and/or at least one micropump and/or at least one electroosmotic pump and/or at least one liquid valve and/or at least one sensoric and/or analytical component.

The components for liquid transport can be entirely or partially composed of a polymer, such as polydimethylsiloxane, polystyrene, polycaprolactone, polycarbonate, PEEK, PTFE, PP, PE, cyclic olefin copolymer, polymethyl methacrylate, polyimide, polyether ether ketone; of glass, silicon, metal, ceramic; and/or of photolithographically structured polymers, such as SU-8, DF3500, ADEX, SUEX, or KMPR photoresist.

It is furthermore advantageous if the at least one microacoustic component and/or the at least one component for liquid transport comprises at least one microfluidic and/or liquid conducting structure element, and the at least one component for liquid transport is connected in a liquid conducting manner to the at least one microacoustic component via at least one liquid conducting structure element.

Advantageously, at least one microchannel and/or one connecting element and/or one sealing element and/or one reservoir and/or one micromembrane and/or one needle-shaped structure element and/or one sponge- or tissue-shaped element can be present as a microfluidic and/or liquid conducting structure element.

Furthermore, according to the invention, at least one region is present on the surface of the at least one microacoustic component on which an acoustofluidic interaction between the acoustic wave and the liquid is realized for the aerosol generation.

In accordance with the solution according to the invention, the liquid which is converted into an aerosol is to be conducted, by means of the at least one microfluidic and/or liquid conducting structure element of the microacoustic component and/or of the component for liquid transport, at least partially into the region on the surface of the microacoustic component, in which region the acoustofluidic interaction between the acoustic wave and liquid is realized.

The at least one microfluidic and/or liquid conducting structure element or structure elements can be arranged at least partially outside of the surface region of the microacoustic component where the acoustofluidic interaction between the acoustic wave and the liquid takes place.

According to the invention, however, at least one opening of the component for liquid transport or of the at least one microfluidic and/or liquid conducting structure element from which the liquid is discharged must be arranged in the region or in the proximity of the surface region of the microacoustic component where the acoustofluidic interaction takes place between the acoustic wave and the liquid. Advantageously, said opening of the component for liquid transport or of the at least one microfluidic and/or liquid conducting structure element is arranged at the point or the region at which a demonstrable increase in the amplitude of the excited wave occurs. Said demonstrable increase in the amplitude can advantageously be 5-30%, more advantageously 5-15% of the maximum amplitude of the wave or waves, or 104 to 10 nm/mm, advantageously 10-3 to 5 nm/mm.

The liquids which are to be converted into an aerosol can be technical inks, conductive or non-conductive liquids with or without nanoparticles, particle-free liquids, liquids with metal particles, liquids which contain biological components such as molecules, DNA, RNA, proteins, enzymes, lipopolymers, sugars, antibodies, vesicles, cells, and other biological components (bioliquids, bioinks) for example, or pure glycerin, resin-based liquids, technical liquids, molten metals or polymers or graphite ink.

Furthermore, according to the invention, at least one component for the transmission of a high-frequency signal is present which is connected in an electrically conductive and high-frequency suitable manner to at least one electrical high-frequency signal line and to the at least one microacoustic component and which realizes the transmission of a high-frequency signal to and/or from the at least one microacoustic component via the at least one electrical high-frequency signal line.

A component of this type for the transmission of a high-frequency signal can advantageously be a printed circuit board, a flexible printed circuit board, or a layered component with integrated electrical lines. The electrically conductive connection to the microacoustic component can advantageously be produced via spring pins, preferably via high-frequency suitable, impedance-matched spring pins, springs, and/or via capacitive or inductive signal coupling.

Furthermore, at least one transport gas component is provided according to the invention. Within the scope of the present invention, a transport gas component is to be understood as a component with which the transport of a gas at least into the region of the acoustofluidic interaction between the acoustic wave and liquid on the surface of the microacoustic component is realized.

The transport gas component is connected to at least one feed and/or removal line for gases, wherein with the transport gas component, at least the feed of at least one gas into the immediate proximity of the region of the acoustofluidic interaction between the acoustic wave and the liquid for the aerosol generation is realized on the surface of the microacoustic component to which the liquid is applied.

Advantageously, the at least one transport gas component realizes the coaxial inflow of a gas into the region of the acoustofluidic interaction between the acoustic wave and the liquid for the purpose of sheathing the generated aerosol stream or jet.

Advantageously, the location of the discharge of the gas from the transport gas component is arranged maximally 30 mm, advantageously <15 mm, more advantageously <5 mm, away from the liquid carrying surface of the at least one microacoustic component in the direction of the volume flow vector of the aerosol stream or jet.

In addition, it is advantageous if the rotationally symmetrical volume flow vector of the coaxially inflowing gas deviates by no more than 45°, advantageously by no more than 30°, from the volume flow vector of the aerosol stream or jet, or from the surface normal of the liquid conducting surface of the at least one microacoustic component.

It is also advantageous if, downstream from the at least one transport gas component or after the at least one opening in the aerosol discharge component in the direction of the volume flow vector of the aerosol stream or jet, at least one further component is present for the aerodynamic focusing into an aerosol stream or jet with a gas sheath and/or for increasing the velocity of the aerosol stream or jet, which component comprises at least one additional feed and/or removal line for gases.

It is advantageous if the further component for the focusing and velocity increase is arranged downstream and, for example, is or contains a filter, a nozzle, or a funnel.

Furthermore, according to the invention at least one component is present for positioning and/or mechanically fixing the at least one component for the transmission of a high-frequency signal and the at least one component for liquid transport in relation to the at least one microacoustic component or to the at least one holder component.

According to the invention, the at least one component for positioning and/or mechanical fixing can thereby be a standalone component and/or a component of the holder component and/or of the component for liquid transport and/or of the aerosol discharge component and/or of another component.

Advantageously, at least one component for positioning and/or mechanical fixing realizes the reversible vertical tilting in the sense of a partial rotation or the reversible vertical raising in the sense of a displacement or the reversible sliding at least of the at least one component for the transmission of a high-frequency signal and of the at least one component for liquid transport in relation to the at least one microacoustic component or to the at least one holder component, in order to be able to realize the replacement of the microacoustic component.

It is furthermore advantageous that the reversible installation of the at least one microacoustic component can be realized by a lateral sliding, insertion, or displacement of the microacoustic component into or onto the holder component, or by a vertical placement or insertion of the microacoustic component into or onto the holder component.

It is also advantageous if at least one open-loop and/or closed-loop control component is present for the open-loop and/or closed-loop control of the discharge of the aerosol stream or jet from the aerosol discharge component or from the component for the aerodynamic focusing of the aerosol stream or jet, which control component is arranged on the outer side or inner side of the aerosol discharge component or of the component for the aerodynamic focusing of the aerosol stream or jet or is arranged downstream above the microacoustic component and/or is part of the component for liquid transport

It is also advantageous if the open-loop and/or closed-loop control component is a rotatable or movable bar that can be positioned in the aerosol stream or jet and/or is a component for closing the opening or the openings and/or is a component which contains a suction device.

In the advantageous case that the open-loop and/or closed-loop control component contains a suction device, the aerosol can thus be collected and removed from the acoustofluidic device.

A great advantage of the solution according to the invention is that the acoustofluidic device for generating aerosols itself has very small dimensions of only a few centimeters in all spatial directions. The low complexity of the individual components enables a simple plug-and-play setup with application-adapted components which can be easily integrated into existing systems and allow shorter installation times, lower maintenance requirements, and reduced costs. High atomization efficiency, small and adjustable droplet sizes, a compact, simple design, and good reproducibility of the aerosol properties make the acoustofluidic device for generating aerosols according to the invention ideal for various applications.

The microacoustic aerosol generation which is used according to the invention is superior to the established atomization methods (ultrasound or pneumatic) in many ways. On the whole, in addition to other advantageous mentioned below, it enables an entirely new, compact design of the device with lower complexity, in which specifically the aerosol generation and the aerosol focusing are integrated in one unit. Components such as an aerosol chamber, aerosol feed lines, and a virtual impactor are no longer necessary. As a result, the space required by the device and the acquisition costs are significantly reduced-one of the most important requirements for access to the market and for practicability.

The simpler design of the acoustofluidic device according to the invention, without aerosol chamber and tubes, also results in a lower expenditure of labor and resources for the setup and cleaning of the device, which lowers the operating costs. The acoustofluidic device according to the invention thus also enables a higher efficiency in the aerosol generation and a better utilization of the liquid that is to be atomized.

Small volumes (<500 μl) of the often very expensive printing liquids suffice for the atomization, and a rapid changeover between different liquids is unproblematic, and the technology provides more accurate control over the volume of the deposited materials, which is advantageous for surface coating processes. In addition, liquids in a very wide viscosity range can be processed, and the temperature control of the acoustofluidic device according to the invention enables a temperature control of the liquid. For an automated aerosol deposition system, the acoustofluidic device according to the invention can easily be integrated into commercially available CAD-compatible production or printing machines, physical and/or chemical syntheses devices, 3D printing platforms, or multi-axis robots.

In addition, it is of particular significance that, with the solution according to the invention, the angle of the aerosol stream or jet can be changed in relation to the surface that is to be coated with the aerosol, by altering the phase difference and/or the phase shift of a waveform, or by the electric power fed to the microacoustic component. The change in the direction of the aerosol stream or jet using the acoustofluidic device according to the invention has even more enhanced effects. For example, the millisecond change in the direction of the aerosol stream or jet can be used as an electronic valve in order to activate or deactivate the atomization process, which is crucial in a printing application. In addition, the control of the atomization direction can also be used to control or to maximize the condensation region of the aerosol on the surface.

The size of the aerosol stream or jet and the starting velocity of the aerosol can also be influenced in situ in order to improve the focusing of the aerosol and the print resolution.

By conducting a gas into the region of the aerosol generation via the at least one transport gas component, a gaseous sheathing of the aerosol, that is, a sheath gas flow, can be realized in said location. By accelerating the aerosol, for example through a subsequent reduction of the outlet diameter and/or through an increase of the sheath gas flow rate, an aerosol stream or jet can be generated which is discharged from the openings of the aerosol discharge component and can realize a targeted application of the aerosol to a surface.

The components according to the invention can be produced using known methods for producing complex micro- or macrostructures, advantageously at least partially with additive manufacturing methods, lithography, or CNC techniques.

A further special advantage of the solution according to the invention is that the liquid used and the aerosol generated therefrom do not need to be returned in a circuit to the acoustofluidic device according to the invention, since the liquid is essentially directly atomized into an aerosol during the acoustofluidic interaction with the acoustic wave and is conducted out of the acoustofluidic device.

This prevents contamination and increases the stability of both the liquid and/or of the liquid composition and also the quality of the aerosol deposition. The effects of external temperature fluctuations can also be prevented or limited, since the microacoustic component, that is, the component which is in contact with the liquid that is to be atomized, can be heated or cooled as needed. The combination with a heat transport component, which can also contain a Peltier element, and with at least one temperature sensor enables a direct monitoring and control of the temperature variations, whereby the compatibility of the acoustofluidic device according to the invention with sensitive liquids, the efficiency, and the liquid quality can be monitored and also improved during use.

In the solution according to the invention, surface acoustic waves are advantageously used in order to atomize liquids into an aerosol of precise droplets with a size of less than 30 μm, which significantly improves the aerosol quality relevant for the deposition.

Through the use of the acoustofluidic device for generating aerosols according to the invention in printing technology, advantages over conventional aerosol sources, inkjet or other print head technologies are obtained, such as for example a significantly improved scalability, cost-efficiency, material compatibility and energy efficiency, aerosol or deposition rate, aerosol resolution, and/or versatility.

The acoustofluidic device according to the invention realizes an atomization of the liquid into a compact aerosol with a small aperture angle, whereby a significantly improved focusing of the aerosol stream can be achieved.

A further advantage of the acoustofluidic device according to the invention is that all components can be arranged in a gas-tight enclosure, for example a standard pipe and flange system, which can prevent pressure drops and the release of nanoparticles and enables a simple integration into synthesis devices.

Further advantages of the solution according to the invention are:
simple replacement of the microacoustic component while simultaneously ensuring a reliable supply of liquid and an electrical high-frequency connection; and
the prevention of the recirculation of the liquid and of the negative impact on the liquid properties due to a longer exposure to the acoustic field.

The invention is explained in greater detail below with the aid of an exemplary embodiment.

In this matter:

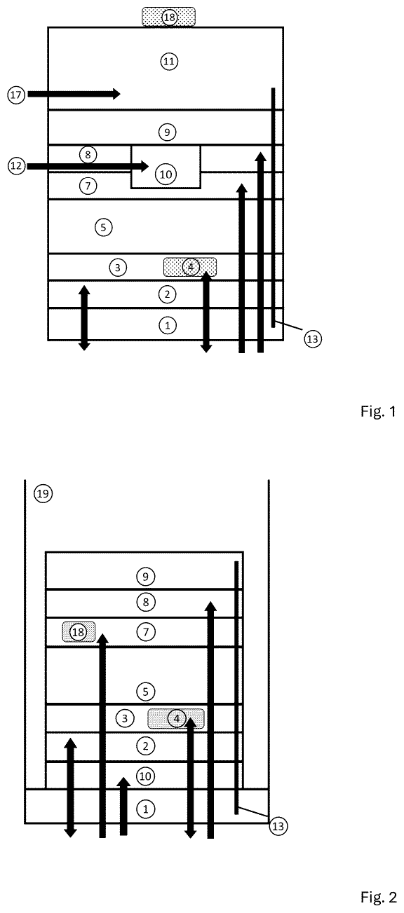

FIG. 1 shows the schematic structure of an acoustofluidic device for generating aerosols according to the invention, for use as a print head; and

FIG. 2 shows the schematic design of an acoustofluidic device for generating aerosols according to the invention, integrated into a tubular chemical synthesis device

### EXAMPLE 1

An acoustofluidic device for generating aerosols according to FIG. 1 comprises a base plate 1 on which all components are positioned. The feed and/or removal lines for gases 12, 17 are thereby laterally connected to the acoustofluidic device, and the electrical high-frequency signal line 6, the electrical signal line 16, the feed and/or removal lines for liquids 15 and for heat 14 are connected to the acoustofluidic device from the rear via the base plate 1.

On the front side of the base plate 1, a component for heat transport 2 is arranged which comprises a cavity filled with cooling liquid and is connected to two feed and removal lines for heat 14, whereby a closed system with two liquid-filled tubes is realized, which system is connected to a peripheral cooler and, in a closed circuit, transports warm cooling liquid from the heat transport component 2 to the cooler and cold cooling liquid to the heat transport component 2.

As a result of this arrangement, the acoustofluidic device is actively cooled by means of a liquid cooling from peripheral components.

The microacoustic component 5 is arranged on the holder component 3 above the component for heat transfer 2.

The microacoustic component 5 is a cartridge which contains a microacoustic chip that is attached by a thermally conductive, double-sided adhesive silicone tape with a thickness of 200 μm as a holder component 3 to a 2-mm thick plate of the microacoustic component 5 of copper. On the microacoustic chip, Rayleigh standing surface acoustic waves (sSAWs) are excited via two opposing interdigital transducers (IDT) (type 24, 90 μm wavelength, 0.5 mm aperture, 6 mm distance apart, adapted to an impedance of 50Ω through the use of 46 finger electrode pairs each). The plate on which the microacoustic chip is positioned is composed of transparent lithium niobate polished on one side (128° YX), and the interdigital transducer electrodes are composed of a layer construction of a 10-nm thick layer of Ti, followed by a 290-nm thick layer of Al.

There are 500 nm of silicon dioxide as a protective layer on the microacoustic chip. On the microacoustic chip is a partially open, droplet-shaped microfluidic liquid conducting structure with an inner channel (channel width=100 μm, wall width at outlet=50 μm) which is composed of two layers of 50-μm thick epoxy dry-film resist (wall layer+cover layer), in order to realize the liquid feed on the surface of the microacoustic chip.

As a component for liquid transport 7, a plate of PEEK plastic is arranged on the microacoustic component 5 and the holder component 3 and is connected in a liquid-tight manner via an O-ring to the partially open, droplet-shaped microfluidic liquid conducting structure of the microacoustic component 5.

A temperature sensor 4 is arranged in a drilled hole inside of the holder component 3 and is connected to a peripheral temperature logger via an electrical signal line 16. The component for liquid transport 7 and the component for the transmission of a high-frequency signal 8 were mounted on top of the holder component 3 and attached with screws, whereby the microacoustic component 5 is connected electrically and in a liquid conducting manner to the electrical signal line 16 and to the feed line for liquid 15.

The component for liquid transport 7 and the component for the transmission of a high-frequency signal 8 thereby have in the center thereof a hole through which the aerosol can exit the acoustofluidic device. A transport gas component 10 is partially attached inside of said hole, in order to transport the transport gas nitrogen via the feed line for gas 12 at a flow rate of 167 sccm and, at a vertical distance of 10 mm from the surface of the microacoustic component 5 to which liquid is applied, to introduce the nitrogen onto the microacoustic chip as a coaxial gas flow around the generated aerosol stream or jet. However, the transport gas component 10 does not impede the discharge of the aerosol thereby.

On the transport gas component 10, an aerosol discharge component 9 with a central hole of 5 mm diameter is attached as an aerosol outlet.

A component for the focusing of the aerosol 11 is arranged thereupon, in order to improve the aerosol guidance and to increase the velocity of the aerosol with the aid of nitrogen via a feed line for gas 17. The aerosol is conveyed out of the acoustofluidic device through a plastic tip mounted on the Luer lock thread. The plastic tip is equipped with an aerosol guide (Nordson EFD Optimum® SmoothFlow™ with an nozzle-tip inner diameter of 800 μm).

An open-loop and/or closed-loop control component 18, which is composed of a bar that can be rotated 45°, is movably attached to the aerosol focusing component 11. It can be rotated from a lateral position into a position which covers the outlet of the nozzle tip and thus prevents the aerosol discharge.

All components are attached to one another with three components for positioning and mechanical fixing 13 and are held together with 3 screws.

The electrical signals are supplied at the operating frequency of the IDTs from a two-channel signal source via two electrical high-frequency signal lines 6 with SMA and MMCX connectors. The liquid is introduced into the component for liquid transport 7, and thus onto the microacoustic component 5, from a peripheral syringe pump over a PTFE tube via the feed line for liquid 15.

The acoustofluidic device thus carries silver ink (JS-426 Novacentrix) (1:24 with DI water) in lines of multiple millimeters in length and a width of ≈130 μm onto a lithium niobate substrate at a deposition rate of 5 mm/s. The atomization occurs at a liquid volume flow rate of 20 μl/min, a power of 4.4 W, and a signal frequency of 42.8 MHz.

### LIST OF REFERENCE NUMERALS

1 Base plate
2 Heat transport component
3 Holder component
4 Temperature sensor
5 Microacoustic component
6 Electrical high-frequency signal line
7 Component for liquid transport
8 Component for the transmission of a high-frequency signal
9 Aerosol discharge component
10 Transport gas component
11 Aerosol focusing component
12 Feed line for gas
13 Component for positioning and/or mechanical fixing
14 Feed and/or removal lines for heat
15 Feed line for liquids
16 Electrical line and signal line
17 Feed line for gas
18 Open-loop and/or closed-loop control component
19 Tubular enclosure

1. An acoustofluidic device for generating aerosols, which at least contains:
at least one aerosol discharge component with at least one opening from which a directed aerosol stream or jet is discharged; and
at least one holder component for at least one microacoustic component; and
at least one feed and/or removal line each for gases, liquids, and heat, at least one electrical line and/or electrical signal line, and at least one electrical high-frequency signal line; and
at least one heat transport component, which realizes the transport of heat from and/or to at least one holder component and which is connected to at least one feed and/or removal line for heat; and
at least one temperature sensor which is connected in a thermally conductive manner to the holder component and/or to the heat transport component and in an electrically conductive manner to at least one electrical high frequency signal line; and
at least one microacoustic component having a lateral dimension of maximally 4×4 cm² with at least one acoustic transducer and/or electroacoustic and/or piezoelectric transducer for the excitation of acoustic waves in the frequency range of 1 to 500 MHz; and
at least one component for the transmission of a high-frequency signal which is connected in an electrically conductive and high-frequency suitable manner to at least one electrical high-frequency signal line and to at least one microacoustic component, and which realizes the transmission of a high-frequency signal to and/or from the at least one microacoustic component via the at least one electrical high-frequency signal line; and
at least one component for liquid transport which is connected in a liquid conducting manner to at least one feed and/or removal line for liquids and to at least the one microacoustic component, and with which at least the feed of liquids to the surface of the at least one microacoustic component is realized at a volume flow rate between 0.1 μl/min and 5 ml/min; and
at least one transport gas component which is connected to at least one feed and/or removal line for gases, wherein with the transport gas component, at least the feed of at least one gas into the immediate proximity of the region of the acoustofluidic interaction between the acoustic wave and the liquid for the aerosol generation is realized on the surface of the microacoustic component to which the liquid is applied; and
at least one component for positioning and/or mechanically fixing the at least one component for the transmission of a high-frequency signal and the at least one component for liquid transport in relation to the at least one microacoustic component or to the at least one holder component, wherein the at least one component for positioning and/or mechanical fixing is a standalone component and/or a component of the holder component and/or of the component for liquid transport and/or of the aerosol discharge component and/or of another component.

2. The acoustofluidic device according to claim 1 in which the at least one microacoustic component realizes the excitation of at least one acoustic wave, advantageously of a Rayleigh surface acoustic wave and/or of a Sezawa surface acoustic wave; and/or of a plate acoustic wave, advantageously of a symmetrical and/or asymmetrical Lamb wave; and/or of a volume acoustic wave, advantageously of a longitudinal and/or shear wave; and/or of a standing surface acoustic wave; and/or of a solid-state and/or liquid-state acoustic wave.

3. The acoustofluidic device according to claim 1 in which the at least one component for liquid transport is connected to at least one liquid pump and/or at least one liquid reservoir, or contains at least one liquid pump and/or at least one liquid reservoir and/or at least one micropump and/or at least one electroosmotic pump and/or at least one liquid valve and/or at least one sensoric and/or analytical component.

4. The acoustofluidic device according to claim 1 in which at least one component for positioning and/or mechanical fixing realizes a reversible vertical tilting in the sense of a partial rotation or a reversible vertical raising in the sense of a displacement or a reversible sliding at least of the at least one component for the transmission of a high-frequency signal and of the at least one component for liquid transport in relation to the at least one microacoustic component or to the at least one holder component for the purpose of replacing the microacoustic component.

5. The acoustofluidic device according to claim 4 in which the reversible installation of the at least one microacoustic component is realized by a lateral sliding, insertion, or displacement of the microacoustic component onto or into the holder component, or by vertical placement or insertion of the microacoustic component onto or into the holder component.

6. The acoustofluidic device according to claim 1 in which at least one heat transport component contains or is connected to at least one heat pipe and/or one cooling body with or without components for improving the gas convection and/or with liquid cooling connected thereto and/or at least one Peltier element having a passive finned cooler with or without components for improving the gas convection and/or at least one Peltier element having a cooling body with liquid cooling connected thereto.

7. The acoustofluidic device according to claim 1 in which the at least one microacoustic component is a microacoustic chip or a microacoustic cartridge, wherein the microacoustic cartridge is at least composed of at least one microacoustic chip on at least one plate or film of a material with high thermal conductivity, and wherein additional layers of highly thermally conductive material are present at least between the chip and plate or film and/or on the side of the plate or film facing away from the chip.

8. The acoustofluidic device according to claim 7 in which a highly thermally conductive connection is realized between the microacoustic chip and the plate or film using an adhesive and/or an adhesion-promoting liquid, and/or single- or double-sided adhesive tape, and/or metal- and/or ceramic-filled polymers, epoxies and/or pastes, and/or an adhesive tape or adhesive strip, and/or by means of a non-permanent adhesive.

9. The acoustofluidic device according to claim 1 in which at least one microacoustic component and/or at least one component for liquid transport comprises at least one microfluidic and/or liquid conducting structure element, and the at least one component for liquid transport is connected in a liquid conducting manner to the microacoustic component via at least one microfluidic and/or liquid conducting structure element.

10. The acoustofluidic device according to claim 9 in which at least one microchannel and/or one connecting element and/or one sealing element and/or one reservoir and/or one micromembrane and/or one needle-shaped structure element and/or one sponge- or tissue-shaped element is present as a microfluidic and/or liquid conducting structure element.

11. The acoustofluidic device according to claim 1 in which at least one transport gas component generates is a coaxial inflow of a gas into the region of the acoustofluidic interaction between the acoustic wave and the liquid for the purpose of sheathing the generated aerosol stream or jet, wherein the site of the discharge of the gas from the transport gas component is arranged maximally 30 mm, advantageously<15 mm, more advantageously<5 mm, away from the liquid carrying surface of the at least one microacoustic component in the direction of the volume flow vector of the aerosol stream or jet.

12. The acoustofluidic device according to claim 11 in which the rotationally symmetrical volume flow vector of the coaxially inflowing gas deviates by no more than 45°, advantageously by no more than 30°, from the volume flow vector of the aerosol stream or jet, or from the surface normal of the liquid carrying surface of the at least one microacoustic component.

13. The acoustofluidic device according to claim 1 in which, downstream from the at least one transport gas component or after the at least one opening in the aerosol discharge component in the direction of the volume flow vector of the aerosol stream or jet, at least one component is present for the aerodynamic focusing of the aerosol stream or jet with a gas sheath and/or for increasing the velocity of the aerosol stream or jet, whereby this component comprises at least one additional feed and/or removal line for gases.

14. The acoustofluidic device according to claim 1 in which an additional, tubular enclosure surrounds the acoustofluidic device, whereby this enclosure is composed of metal, polymer, ceramic, or glass and which comprises on at least one tube end at least one flange of the type ISO-K, ISO-KF, ISO-F, CF, or COF for the feed and removal lines and electrical signal lines and electrical high-frequency signal lines.

15. The acoustofluidic device according to claim 1 in which the opening or the openings in the aerosol discharge component have dimensions of no greater than 5 mm, and/or have a circular, coaxial, and/or oval shape, and/or are multi-opening outlets, and/or have Luer threads and/or metric threads and/or inch threads.

16. The acoustofluidic device according to claim 1 in which at least one open-loop and/or closed-loop control component is present for the open-loop and/or closed-loop control of the discharge of the aerosol stream or jet from the aerosol discharge component or from the component for the aerodynamic focusing of the aerosol stream or jet, which control component is arranged on the outer side or inner side of the aerosol discharge component or of the component for the aerodynamic focusing of the aerosol stream or jet or is arranged downstream above the microacoustic component and/or is part of the component for liquid transport.

17. The acoustofluidic device according to claim 16 in which the open-loop and/or closed-loop control component is a rotatable or movable bar that is positioned in the aerosol stream or jet and/or is a component for closing the opening or the openings and/or is a component which contains a suction device.
